(19) United States
(12) Patent Application Publication
(10) Pub. No.: US 2023/0309889 A1
(43) Pub. Date: Oct. 5, 2023

(54) Garment with Electrode

(71) Applicant: Nippon Telegraph and Telephone Corporation, Tokyo (JP)

(72) Inventors: Takako Ishihara, Tokyo (JP); Nobuaki Matsuura, Tokyo (JP); Kei Kuwabara, Tokyo (JP); Hiroto Matsuoka, Tokyo (JP); Yuichi Higuchi, Tokyo (JP); Hiroyoshi Togo, Tokyo (JP)

(21) Appl. No.: 18/006,691

(22) PCT Filed: Aug. 20, 2020
(86) PCT No.: PCT/JP2020/031431

§ 371 Date: Jan. 24, 2023

(51) Int. Cl. A61B 5/256; A61B 5/28; A41D 13/12

(52) U.S. Cl. CPC A61B 5/256; A61B 5/28; A41D 13/1281

(57) ABSTRACT

A garment with electrodes includes a different electrode disposed in the vicinity of a left shoulder of a garment on a lining of a back body of the garment so as to be in contact with an inclined surface of a raised portion of a left shoulder blade of a back of a subject to be measured; an indifferent electrode disposed in the vicinity of a right shoulder of the garment so as to be in contact with an inclined surface of a raised portion of a right shoulder blade of the back of the subject to be measured; a wiring part connected to each of the different electrode and the indifferent electrode; and a controller connected to the wiring part.

### CROSS-REFERENCE TO RELATED APPLICATIONS

This application is a national phase entry of PCT Application No. PCT/JP2020/031431, filed on Aug. 20, 2020, which application is hereby incorporated herein by reference.

### TECHNICAL FIELD

The present invention relates to a garment with electrodes that acquires biological information with high accuracy .

### BACKGROUND

In recent years, in the field of health and medical care, there is growing interest in technology that uses wearable bioelectrodes (sensors) to measure daily health conditions. In particular, long-term measurement and analysis of electrocardiographic signals (electrocardiogram) and the like is known to be effective in the early detection of a heart disease and the like. In long-term measurement of electrocardiograms and the like, a garment equipped with wearable bioelectrodes (garment with electrodes) is attracting attention because users (subjects to be measured) can easily measure electrocardiograms and the like on a daily basis by wearing said garment.[NPL 1]

### CITATION LIST

### Non Patent Literature

[NPL 1] “A Wearable Sensor Developed through Cross-industrial Collaboration,” NTT Technical Review, 2014.5, p42-45.

### SUMMARY

### Technical Problem

In the measurement of biological signals such as electrocardiographic signals in the thorax, abdomen, axilla and back, in order to enhance the accuracy of the measurement of the electrocardiographic signals and the like using a garment with electrodes, it is necessary to closely attach the electrodes to the body surfaces to increase the contact pressure. Therefore, if the contact pressure of the garment with electrodes is low, the electrodes do not attach closely to the body surfaces and noise is generated, making it difficult to acquire biological signals with high accuracy.

The garment with the electrodes also requires a function or instrument for strongly tightening the upper body to closely attach the electrodes to the body surfaces. As a result, a problem arises that the configuration of the garment with electrodes is limited, and the costs increase. Another problem is that the user may experience a feeling of pressure when wearing the garment with electrodes.

### Solution to Problem

In order to solve the problems described above, a garment with electrodes according to embodiments of the present invention includes a different electrode disposed in the vicinity of a left shoulder of a lining of a back body of the garment so as to be in contact with an inclined surface of a raised portion of a left shoulder blade of a back of a subject to be measured, an indifferent electrode disposed in the vicinity of a right shoulder of the lining of the back body of the garment so as to be in contact with an inclined surface of a raised portion of a right shoulder blade of the back of the subject to be measured, a wiring part connected to each of the different electrode and the indifferent electrode, and a controller connected to the wiring part.

The garment with electrodes according to embodiments of the present invention includes: a different electrode disposed in the vicinity of a left shoulder of a lining of a front body of the garment so as to be in contact with an inclined surface of a raised portion of a greater pectoral muscle of a subject to be measured, an indifferent electrode disposed in the vicinity of a right shoulder of the lining of the front body of the garment so as to be in contact with the inclined surface of the raised portion of the greater pectoral muscle of the subject to be measured, a wiring part connected to each of the different electrode and the indifferent electrode, and a controller connected to the wiring part.

### Advantageous Effects of Embodiments of Invention

According to embodiments of the present invention, a garment with electrodes that acquires biological information such as electrocardiographic signals with high accuracy by maintaining adhesion between the electrodes and the body surfaces can be provided.

### BRIEF DESCRIPTION OF THE DRAWINGS

FIG. 1 is a rear view of a garment with electrodes according to a first embodiment of the present invention.

FIG. 2 is a diagram for explaining attachment of the electrodes according to the first embodiment of the present invention.

FIG. 3 shows heart rate data acquired by the garment with electrodes according to the first embodiment of the present invention.

FIG. 4 is a rear view of a garment with electrodes according to a second embodiment of the present invention.

FIG. 5 is a front view of a garment with electrodes according to a third embodiment of the present invention.

FIG. 6 is a diagram for explaining attachment of the electrodes according to the third embodiment of the present invention.

FIG. 7A is a cross-sectional view where a garment with electrodes according to a fourth embodiment of the present invention is worn.

FIG. 7B is a cross-sectional view where a conventional garment with electrodes is worn.

FIG. 8 is a diagram showing an example of a configuration of a computer according to an embodiment of the present invention.

### DETAILED DESCRIPTION OF ILLUSTRATIVE EMBODIMENTS

### First Embodiment

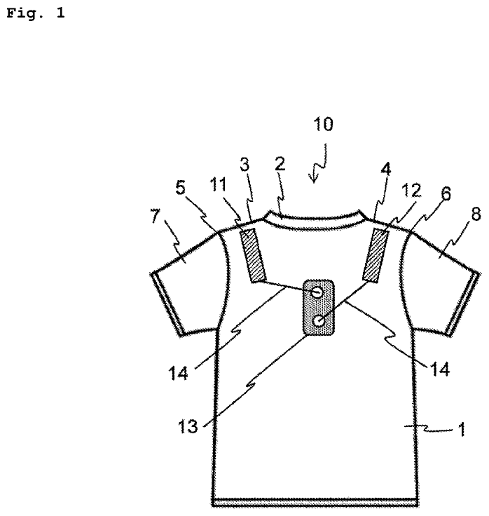

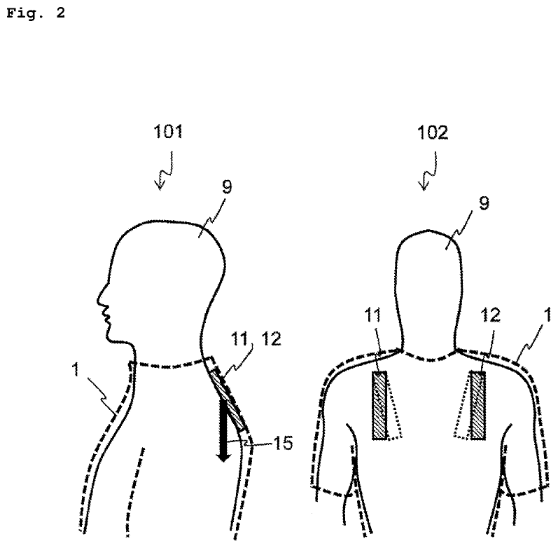

A garment with electrodes according to a first embodiment of the present invention will be described with reference to FIGS. 1 and 2.

### Configuration of Garment with Electrode

FIG. 1 shows a rear view of a garment with electrodes 10 according to the present embodiment. The garment with electrodes 10 includes a garment 1, a different electrode 11, an indifferent electrode 12, a controller 13, and wiring parts 14.

The garment 1 includes, on the left-hand side of a collar 2, a left shoulder 3, a left sleeve 7, and a left sleeve cap 5 at a base end of the left sleeve, and, on the right-hand side from the collar 2, a right shoulder 4, a right sleeve 8, and a right sleeve cap 6 at a base end of the right sleeve.

The garment 1 is a shirt, a T-shirt, a training wear or the like, and common under garment material for T-shirts and the like such as polyester and cotton is used.

The different electrode 11 and the indifferent electrode 12 are electrodes for acquiring electrocardiographic signals, are fixed to linings of the garment 1 respectively, and acquire electrocardiographic signals by coming into contact with skin of a human body. Here, the different electrode 11 is disposed on the left side in the vicinity of the heart, and the indifferent electrode 12 is disposed on the right side.

The different electrode 11 is disposed in a back body of the garment 1 so as to extend downward in a substantially vertical direction from the vicinity a center of the left shoulder 3, that is, in the vicinity of an intermediate point between the left side of the collar 2 and the left sleeve cap 5. More specifically, the different electrode 11 is disposed so as to extend downward in the substantially vertical direction from a position approximately 5 cm away from the left sleeve cap 5 in the left shoulder 3.

The indifferent electrode 12 is disposed in the back body of the garment 1 so as to extend downward in the vertical direction from a center of the right shoulder 4, that is, in the vicinity of an intermediate point between the right side of the collar 2 and the right sleeve cap 6. More specifically, the indifferent electrode 12 is disposed so as to extend downward in the substantially vertical direction from a position approximately 5 cm away from the right sleeve cap 6 in the right shoulder 4.

The size of the different electrode 11 and the indifferent electrode 12 is approximately 5 cm to 7 cm in length and approximately 2 cm in width. Although the present embodiment shows an example in which the different electrode 11 and the indifferent electrode 12 are arranged symmetrically in the garment 1, the different electrode 11 and the indifferent electrode 12 do not have to be symmetrical.

A conductive fibrous material is used for the different electrode 11 and the indifferent electrode 12. As the conductive fibrous material, for example, a fiber material containing a polymer or a metal having conductivity, such as a material obtained by forming a film of metal on a fiber by plating or vapor deposition or a material obtained by knitting a metallic yarn into a fiber can be used. As the metal, silver (Ag), aluminum (Al) or the like can be used.

The different electrode 11 and the indifferent electrode 12 are fixed to the garment 1 by being sewn or adhered to the garment 1.

The different electrode 11 and the indifferent electrode 12 are electrically connected to the controller 13 through the respective wiring parts 14. Here, a conductive fibrous material is used as the material for the wiring parts 14, as with the different electrode 11 and the indifferent electrode 12.

An electrocardiographic signal acquired as a potential difference between the different electrode 11 and the indifferent electrode 12 is transmitted to the controller 13. The controller 13 may be provided with a power source.

According to this configuration, electrocardiographic signals acquired in the different electrode 11 and the indifferent 12 are transmitted to the controller 13 via the wiring parts 14. The controller 13 may be provided with a storage unit, and the electrocardiographic signals to be transmitted may be stored in the storage unit. Alternatively, the controller 13 may be provided with a transmission unit, and the electrocardiographic signals to be transmitted may be transmitted to a smart phone or a server, processed, and stored.

FIG. 2 shows a left side view 101 and a rear view 102 in a mode in which a user (a subject to be measured) wears the garment with electrodes 10 according to the present embodiment.

When the garment with electrodes 10 according to the present embodiment is worn, the different electrode 11 and the indifferent electrode 12 are attached closely to the body surfaces of the user (subject to be measured) regardless of the posture of the user (subject to be measured), due to a dead weight 15 of the garment 1 such as a shirt, so that electrocardiographic signals can be favorably acquired even in a state where the contact pressure is low due to wearing a garment with low adhesion such as a shirt.

More specifically, when a user (a subject to be measured) 9 wears the garment with electrodes 10 according to the present embodiment, the different electrode 11 is attached to the body surface on the back so as to extend from a portion below the collarbone in the vicinity of the left shoulder toward a raised portion of the left shoulder blade. Similarly, the indifferent electrode 12 is attached to the body surface on the back so as to extend from a portion below the collarbone in the vicinity of the right shoulder toward a raised portion of the right shoulder blade.

As a result, the different electrode 11 and the indifferent electrode 12 are attached so as to be in contact with inclined surfaces of the raised portions of the left and right shoulder blades, respectively, and therefore are attached closely to the raised portions of the shoulder blades by the dead weight 15 of the garment 1.

In the acquisition of electrocardiographic signals, in order to avoid noises caused by a movement of a collarbone, when the garment with electrodes 10 is worn, the different electrode 11 and the indifferent electrode 12 are attached so as to extend downward from the body surfaces of the portions below the collarbones so as not to overlap with the collarbones.

In particular, clothing (garment) is apt to be wrinkled when the arms move back and forth as in a daily walking motion of the user. While the adhesion between the electrodes and the body surfaces is reduced in the conventional technology, the different electrode 11 and the indifferent electrode 12 are attached to the raised portions of the shoulder blades in the garment with electrodes 10 according to the present embodiment, thereby maintaining stable adhesion therebetween.

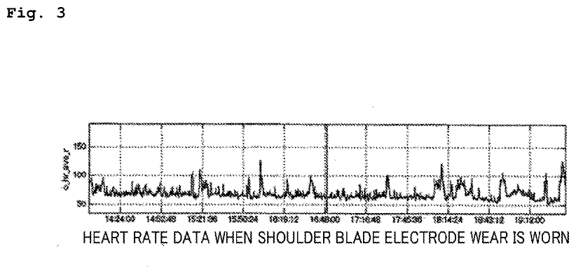

As a result of measuring an electrocardiogram by having the garment with electrodes 10 according to the present embodiment worn, as shown in FIG. 3, it is possible to measure a favorable electrocardiogram with less noise, which is equivalent to an electrocardiogram measured by closely attaching the different electrode and the indifferent electrode to the body surfaces.

As described above, according to the garment with electrodes 10 according to the present embodiment, the different electrode 11 and the indifferent electrode 12 can be attached closely to the body surfaces by the dead weight 15 of the garment 1, so that electrocardiographic signals with low noise can be acquired with high accuracy.

Further, since there is no need for a mechanism to strongly tighten the upper body to increase the contact pressure, electrocardiographic signals can be acquired with a simple configuration at low cost.

The user (subject to be measured) can easily wear the garment with electrodes 10 according to the present embodiment without experiencing a feeling of pressure.

Although the present embodiment shows an example in which the different electrode 11 and the indifferent electrode 12 are disposed in the back body of the garment 1 so as to extend downward in the substantially vertical direction from the vicinities of the centers of the left and right shoulders, the present invention is not limited thereto. The different electrode 11 may be disposed in the vicinity of the left shoulder 3 of the garment 1 so as to be in contact with the inclined surface of the raised portion of the left shoulder blade. The indifferent electrode 12 may be disposed in the vicinity of the right shoulder 4 of the garment 1 so as to be in contact with the inclined surface of the raised portion of the right shoulder blade.

### Second Embodiment

Next, a second embodiment of the present invention will be described. A garment with electrodes according to the second embodiment has substantially the same configuration and exhibits substantially the same effects as the garment with electrodes according to the first embodiment, but differs in the arrangement of the different electrode and the indifferent electrode.

### Configuration of Garment with Electrodes

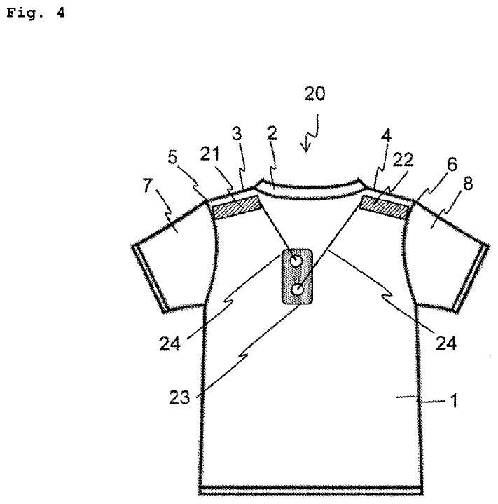

FIG. 4 shows a rear view of a garment with electrodes 20 according to the present embodiment. The garment with electrodes 20 includes a garment 1, a different electrode 21, an indifferent electrode 22, a controller 23, and wiring parts 24.

The different electrode 21 is disposed in the back body of the garment 1 so as to extend from the left side of the collar 2 toward the left sleeve cap 5, in the vicinity of the left shoulder 3, that is, specifically, in a section approximately 2 to 3 cm below the left shoulder 3.

The indifferent electrode 22 is disposed in the back body of the garment 1 so as to extend from the right side of the collar 2 toward the right sleeve cap 6, in the vicinity of the right shoulder 4, that is, specifically, in a section approximately 2 to 3 cm below the right shoulder 4.

When the garment with electrodes 20 according to the present embodiment is worn, the bioelectrodes are attached closely to the body surfaces of a user (a subject to be measured) regardless of the posture of the user (subject to be measured), due to the dead weight 15 of the garment such as a shirt, so that electrocardiographic signals can be favorably acquired even in a state where the contact pressure is low due to wearing a garment with low adhesion such as a shirt.

More specifically, when the user (subject to be measured) wears the garment with electrodes 20 according to the present embodiment, the different electrode 21 and the indifferent electrode 22 are attached to the body surfaces on the back, extending toward the acromions in the upper trapezius muscle.

As a result, the different electrode 21 and the indifferent electrode 22 are attached so as to be in contact with the raised portions (inclined surfaces) of the shoulder blades, and therefore are closely attached to the raised portions (inclined surfaces) of the shoulder blades by the dead weight 15 of the garment 1. Therefore, the electrocardiographic signals can be favorably acquired even in a state where the contact pressure is low due to wearing a garment with low adhesion such as a shirt.

Further, when the garment with electrodes 10 according to the first embodiment is worn, the different electrode 21 and the indifferent electrode 22 are disposed on the shoulder straps of underwear such as a brassiere, making it impossible to acquire the electrocardiographic signals satisfactorily.

However, when the garment with electrodes 20 according to the present embodiment is worn, the different electrode 21 and the indifferent electrode 22 are disposed so as to cross over the shoulder straps of underwear such as a brassiere, allowing for close attachment of the electrodes to the body surfaces and allowing for favorable acquisition of electrocardiographic signals.

As a result of measuring an electrocardiogram by having the garment with electrodes 20 according to the present embodiment worn, it is possible to measure a favorable electrocardiogram with less noise, which is equivalent to the electrocardiogram measured by closely attaching the different electrode and the indifferent electrode to the body surfaces.

### Third Embodiment

Next, a third embodiment of the present invention will be described. A garment with electrodes according to the third embodiment has substantially the same configuration and exhibits substantially the same effects as the garment with electrodes according to the first embodiment, but differs in that the different electrode and the indifferent electrode are disposed in the front body of the garment.

### Configuration of Garment with Electrodes

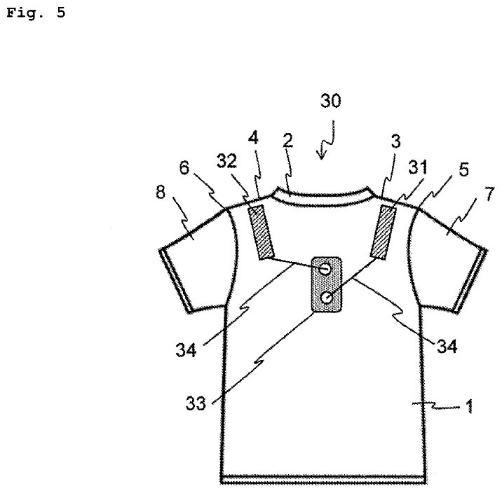

FIG. 5 shows a front view of a garment with electrodes 30 according to the present embodiment. The garment with electrodes 30 includes a garment 1, a different electrode 31, an indifferent electrode 32, a controller 33, and wiring parts 34.

The different electrode 31 and the indifferent electrode 32 are electrodes for acquiring electrocardiographic signals, are fixed to linings of the garment 1 respectively, and acquire electrocardiographic signals by coming into contact with skin of a human body. Here, the different electrode 31 is disposed on the left side in the vicinity of the heart, and the indifferent electrode 32 is disposed on the right side.

The different electrode 31 is disposed in the front body of the garment 1 so as to extend downward in the substantially vertical direction from the vicinity of the center of the left shoulder 3, that is, from the vicinity of the intermediate point between the left side of the collar 2 and the left sleeve cap 5. More specifically, the different electrode 11 is disposed so as to extend downward in the substantially vertical direction from a position approximately 5 cm away from the left sleeve cap 5 in the left shoulder 3.

The indifferent electrode 32 is disposed in the front body of the garment 1 so as to extend downward in the substantially vertical direction from the center of the right shoulder 4, that is, from the vicinity of the intermediate point between the right side of the collar 2 and the right sleeve cap 6. More specifically, the indifferent electrode 12 is disposed so as to extend downward in the substantially vertical direction from a position approximately 5 cm away from the right sleeve cap 6 in the right shoulder 4.

The size of the different electrode 31 and the indifferent electrode 32 is approximately 5 cm to 7 cm in length and approximately 2 cm in width. Although the present embodiment shows an example in which the different electrode 31 and the indifferent electrode 32 are arranged symmetrically in the garment 1, the different electrode 31 and the indifferent electrode 32 do not have to be symmetrical.

The different electrode 31 and the indifferent electrode 32 are fixed to the garment 1 by being sewn or adhered to the garment 1.

The different electrode 31 and the indifferent electrode 32 are electrically connected to the controller 33 through the respective wiring parts 34. Here, a conductive fibrous material is used as the material for the wiring parts 34, as with the different electrode 31 and the indifferent electrode 32.

An electrocardiographic signal acquired as a potential difference between the different electrode 31 and the indifferent electrode 32 is transmitted to the controller 33. The controller 33 may be provided with a power source.

According to this configuration, electrocardiographic signals acquired in the different electrode 31 and the indifferent 32 are transmitted to the controller 33 via the wiring parts 34. The controller 33 may be provided with a storage unit, and the electrocardiographic signals to be transmitted may be stored in the storage unit. Alternatively, the controller 33 may be provided with a transmission unit, and the electrocardiographic signals to be transmitted may be transmitted to a smart phone or a server, processed, and stored.

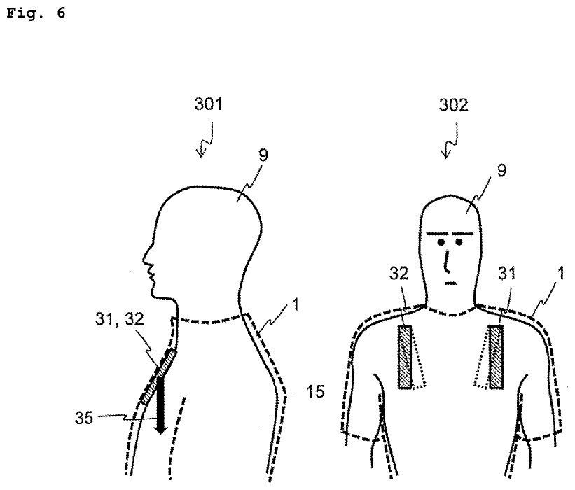

FIG. 6 shows a left side view 301 and a front view 302 in a mode in which a user (a subject to be measured) wears the garment with electrodes 30 according to the present embodiment.

When the garment with electrodes 30 according to the present embodiment is worn, the different electrode 31 and the indifferent electrode 32 are attached closely to the body surfaces of the user (subject to be measured) regardless of the posture of the user (subject to be measured), due to a dead weight 35 of the garment 1 such as a shirt, so that electrocardiographic signals can be favorably acquired even in a state where the contact pressure is low due to wearing a garment with low adhesion such as a shirt.

More specifically, when the user (subject to be measured) 9 wears the garment with electrodes 30 according to the present embodiment, the different electrode 31 is attached to the body surface on the chest so as to extend from the collarbone in the vicinity of the left shoulder toward the greater pectoral muscle of the left chest. Similarly, the indifferent electrode 32 is attached to the body surface on the chest so as to extend from the collarbone in the vicinity of the right shoulder toward the greater pectoral muscle of the right chest.

As a result, the different electrode 31 and the indifferent electrode 32 are attached so as to be in contact with the inclined surfaces of the raised portions of the left and right greater pectoral muscle respectively, and therefore are closely attached to the raised portions of the shoulder blades by the dead weight 35 of the garment 1.

In the acquisition of electrocardiographic signals, in order to avoid noises caused by a movement of a collarbone, when the garment with electrodes 30 is worn, noises can be avoided by covering parts of the different electrode 31 and the indifferent electrode 32 overlapping with the collarbones with a non-conductive material.

Further, clothing (garment) is apt to be wrinkled when the arms move back and forth as in a daily walking motion of the user. While the adhesion between the electrodes and the body surfaces is reduced in the conventional technology, the different electrode 31 and the indifferent electrode 32 are attached to the raised portions of the greater pectoral muscle in the garment with electrodes 30 according to the present embodiment, thereby maintaining stable adhesion therebetween.

As a result of measuring an electrocardiogram by having the garment with electrodes according to the present embodiment worn, it is possible to measure a favorable electrocardiogram with less noise, which is equivalent to the electrocardiogram measured by closely attaching the different electrode and the indifferent electrode to the body surfaces.

In particular, in the garment with electrodes 30 according to the present embodiment, by disposing the different electrode 31 and the indifferent electrode 32 in the front body of the garment 1, a higher cardiac potential can be measured compared to when the different electrode 31 and the indifferent electrode 32 are disposed in the back body, thereby acquiring favorable electrocardiographic signals more resistant to noise.

As described above, according to the garment with electrodes 30 according to the present embodiment, the different electrode 31 and the indifferent electrode 32 can be attached closely to the body surfaces by the dead weight 35 of the garment 1, so that electrocardiographic signals with low noise can be acquired with high accuracy.

### Fourth Embodiment

Next, a fourth embodiment of the present invention will be described. A garment with electrodes according to the fourth embodiment has substantially the same configuration and exhibits substantially the same effects as the garment with electrodes according to the first embodiment, but differ in the mode of attachment of the different electrode and the indifferent electrode.

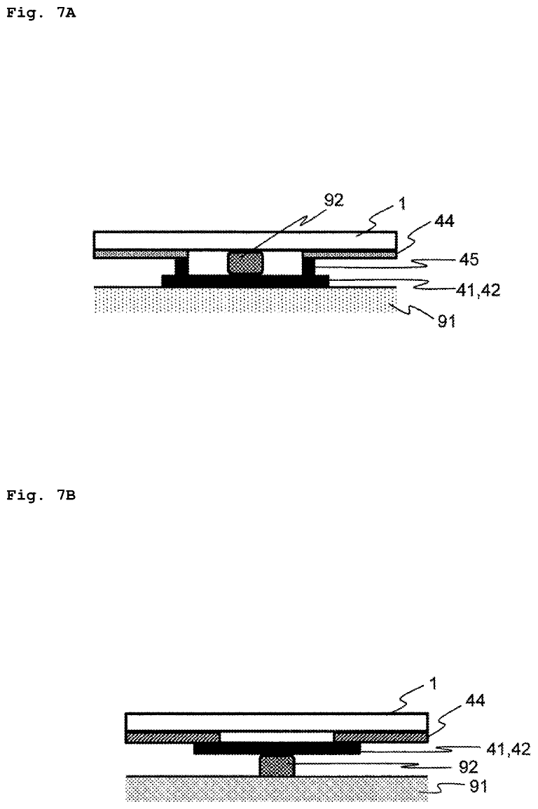

FIG. 7A is a cross-sectional view of the periphery of the different electrode and the indifferent electrode when a garment with electrodes 40 according to the fourth embodiment is worn.

The cross-sectional view shows the cross section in a plane parallel to the shoulders 3 and 4 in the different electrode and the indifferent electrode when the garment with electrodes 40 is worn. For example, in the case of the garment with electrodes 10 according to the first embodiment, the cross section in a width direction is shown, and in the case of the garment with electrodes 20 according to the second embodiment, the cross section in a longitudinal direction is shown.

For comparison, FIG. 7B shows a cross-sectional view of the periphery of the different electrode and the indifferent electrode when the conventional garment with electrodes is worn.

In the garment with electrodes 40, electric wires 44 disposed on an inner surface of the garment 1 are connected to the different electrode 41 and the indifferent electrode 42 through connection members 45. The different electrode 41 and the indifferent electrode 42 are closely attached to skin of the user (subject to be measured), to acquire electrocardiographic signals.

Here, the connection members 45 are electrically conductive, and, for example, a snap, a conductive adhesive tape, a conductive double-sided tape or the like can be used. The connection members 45 allow for the attachment/detachment of the different electrode 41 and the indifferent electrode 42.

When the user (subject to be measured) wears the garment with electrodes 40, the different electrode 41 and the indifferent electrode 42 are removed from the wiring palls 44 beforehand, shoulder straps 92 of underwear such as a brassiere are disposed inside the garment 1, and thereafter the different electrode 41 and the indifferent electrode 42 are fixed so as to cover the shoulder straps 92 using the connection members 45.

When wearing the conventional garment with electrodes, since the shoulder straps 92 are disposed between the different electrode 41 and the indifferent electrode 42 and the skin of the user (subject to be measured), the different electrode 41 and the indifferent electrode 42 cannot be closely attached to the skin of the user (subject to be measured), and therefore electrocardiographic signals with low noise cannot be acquired with high accuracy.

On the other hand, when wearing the garment with electrodes 40 according to the present embodiment, since the shoulder straps 92 are disposed between the garment 1 and the different electrode 41 and the indifferent electrode 42 and are not disposed between the different electrode 41 and the indifferent electrode 42 and the skin of the user (subject to be measured), the different electrode 41 and the indifferent electrode 42 can be closely attached to the skin of the user (subject to be measured), and therefore electrocardiographic signals with low noise can be acquired with high accuracy.

As described above, according to the garment with electrodes 40 according to the present embodiment, the shoulder straps 92 are configured to pass between the garment 1 and the different electrode 41 and the indifferent electrode 42, so that interference between the different electrode 41 and the indifferent electrode 42 and the shoulder straps 92 can be avoided.

As a result, even when the user (subject to be measured) wears underwear such as a brassiere, electrocardiographic signals with low noise can be acquired with high accuracy.

In the present embodiment, a seam tape may be used for fixing the different electrode 41 and the indifferent electrode 42.

The configuration described in the present embodiment can be used in the garments with electrodes according to the first to third embodiments.

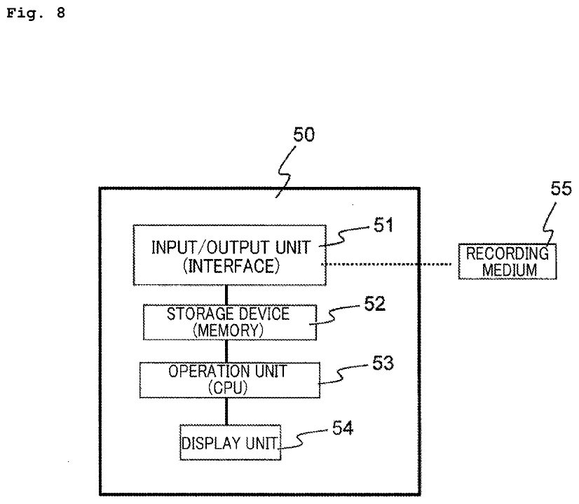

FIG. 8 shows an example of a configuration of a computer 50 in a controller of a garment with electrodes according to an embodiment of the present invention. The controller of the garment with electrodes can be implemented by the computer 50 that includes a CPU (Central Processing Unit) 53, a storage device (storage unit) 52, and an interface device 51, and a program that controls these hardware resources. The CPU 53 executes processing described in the embodiments of the present invention in accordance with the program stored in the storage device 52.

In the controller of the garments with electrodes according to the embodiments of the present invention, the computer 50 may be provided inside the controller, or at least part of the functions of the computer may be implemented by using an external computer. Also, the storage device may also use a storage medium 55 that is external to the device, and a program stored in the storage medium 55 may be read and executed. Examples of the storage medium 55 include various magnetic recording media, a magneto-optic recording medium, a CD-ROM, a CD-R, and various memories. Also, the program may be supplied to the computer via a communication line such as the Internet. When a server, a smart phone or the like is used as an external computer or storage device, data such as electrocardiographic signals can be transmitted wirelessly by providing a transmission function in the controller.

In the embodiments of the present invention, the garments are illustrated shirts with sleeves, but the garments may be shirt without sleeves.

The embodiments of the present invention described examples of the structure, size, material, and the like of each component are shown in the configurations and the like of the garments with electrodes, but are not limited thereto. The configurations of the garments with electrodes may have any configurations as long as the functions and effects of the garments with electrodes are exhibited.

### INDUSTRIAL APPLICABILITY

Embodiments of the present invention can be applied as a wearable device/instrument used for monitoring daily biological information in the field of health medical care.

### REFERENCE SIGNS LIST

1 Garment (back body)

2 Collar

3 Left shoulder

4 Right shoulder

5 Left sleeve cap

6 Right sleeve cap

10 Garment with electrodes

11 Different electrode

12 Indifferent electrode

13 Controller

14 Wiring part.

1-8. (canceled)

9. A garment with electrodes, comprising:
a different electrode disposed in a vicinity of a left shoulder of a lining of a back body of the garment and configured to be in contact with an inclined surface of a raised portion of a left shoulder blade of a back of a subject to be measured;
an indifferent electrode disposed in a vicinity of a right shoulder of the lining of the back body of the garment and configured to be in contact with an inclined surface of a raised portion of a right shoulder blade of the back of the subject to be measured;
a wiring part connected to each of the different electrode and the indifferent electrode; and
a controller connected to the wiring part.

10. The garment with electrodes according to claim 9, wherein:
the different electrode is disposed so as to extend downward in a substantially vertical direction from the vicinity of a center of the left shoulder of the garment; and
the indifferent electrode is disposed so as to extend downward in a substantially vertical direction from the vicinity of a center of the right shoulder of the garment.

11. The garment with electrodes according to claim 9, wherein:
the different electrode is disposed so as to extend from a left-hand side of a collar toward a left sleeve cap in the vicinity of the left shoulder of the garment; and
the indifferent electrode is disposed so as to extend from a right-hand side of the collar toward a right sleeve cap in the vicinity of the right shoulder of the garment.

12. The garment with electrodes according to claim 9, wherein the different electrode and the indifferent electrode are disposed so as not to overlap with collarbones of the subject to be measured.

13. The garment with electrodes according to claim 9, wherein the different electrode and the indifferent electrode are each detachably fixed to the wiring part via a conductive connection member.

14. The garment with electrodes according to claim 9, wherein a potential difference between the different electrode and the indifferent electrode is configured to be acquired as an electrocardiographic signal.

15. A garment with electrodes includes:
a different electrode disposed in a vicinity of a left shoulder of a lining of a front body of the garment and configured to be in contact with an inclined surface of a raised portion of a greater pectoral muscle of a subject to be measured;
an indifferent electrode disposed in a vicinity of a right shoulder of the lining of the front body of the garment and configured to be in contact with the inclined surface of the raised portion of the greater pectoral muscle of the subject to be measured;
a wiring part connected to each of the different electrode and the indifferent electrode; and
a controller connected to the wiring part.

16. The garment with electrodes according to claim 15, wherein portions of the different electrode and the indifferent electrode configured to overlap with collarbones of the subject to be measured are covered with a non-conductive material.

17. The garment with electrodes according to claim 15, wherein the different electrode and the indifferent electrode are each detachably fixed to the wiring part via a conductive connection member.

18. The garment with electrodes according to claim 15, wherein a potential difference between the different electrode and the indifferent electrode is configured to be acquired as an electrocardiographic signal.

19. A garment with electrodes, comprising:
a different electrode disposed in a vicinity of a first shoulder of a lining of a back body of the garment and configured to be in contact with an inclined surface of a raised portion of a first shoulder blade of a back of a subject to be measured;
an indifferent electrode disposed in a vicinity of a second shoulder of the lining of the back body of the garment and configured to be in contact with an inclined surface of a raised portion of a second shoulder blade of the back of the subject to be measured;
a wiring part connected to each of the different electrode and the indifferent electrode; and
a controller connected to the wiring part.

20. The garment with electrodes according to claim 19, wherein:
the different electrode is disposed so as to extend downward in a substantially vertical direction from the vicinity of a center of the first shoulder of the garment; and
the indifferent electrode is disposed so as to extend downward in a substantially vertical direction from the vicinity of a center of the second shoulder of the garment.

21. The garment with electrodes according to claim 19, wherein:
the different electrode is disposed so as to extend from a first-hand side of a collar toward a first sleeve cap in the vicinity of the first shoulder of the garment; and
the indifferent electrode is disposed so as to extend from a second-hand side of the collar toward a second sleeve cap in the vicinity of the second shoulder of the garment.

22. The garment with electrodes according to claim 19, wherein the different electrode and the indifferent electrode are disposed so as not to overlap with collarbones of the subject to be measured.

23. The garment with electrodes according to claim 19, wherein the different electrode and the indifferent electrode are each detachably fixed to the wiring part via a conductive connection member.

24. The garment with electrodes according to claim 19, wherein a potential difference between the different electrode and the indifferent electrode is configured to be acquired as an electrocardiographic signal.
